(19) United States
(12) Patent Application Publication
(10) Pub. No.: US 2020/0061146 A1
(43) Pub. Date: Feb. 27, 2020

(54) METHOD FOR PREPARING CURCUMA XANTHORRHIZA ROXB. EXTRACT

(71) Applicant: COSMAX NS, INC., Seoul (KR)

(72) Inventors: Jin Hak Kim, Seoul (KR); Jae Kwan Hwang, Seoul (KR); Su Young Choi, Seoul (KR)

(21) Appl. No.: 16/461,613

(22) PCT Filed: Nov. 2, 2017
(86) PCT No.: PCT/KR2017/012331

§ 371 Date: May 16, 2019

(30) Foreign Application Priority Data
Nov. 18, 2016 (KR) 10-2016-0154173

(51) Int. Cl. A61K 36/9066

(52) U.S. Cl. CPC A61K 36/9066; A61K 2236/31; A61K 2236/37; A61K 2236/39

(57) ABSTRACT

The present invention relates to a method for preparing a Curcuma xanthorrhiza Roxb. extract. Further, the present invention provides a method for preparing a Curcuma xanthorrhiza Roxb. extract comprising the following steps of: subjecting Curcuma xanthorrhiza Roxb. to supercritical extraction to obtain an extract containing xanthorrhizol as an active ingredient; subjecting the supercritical extraction residue to ethanol extraction or supercritical extraction to obtain an extract containing curcumin as an active ingredient; subjecting the ethanol extraction or supercritical extraction residue to enzyme treatment to obtain immune polysaccharides having average molecular weight 33,000 Da; and obtaining a insoluble dietary fiber from the enzyme treatment residue, an extract containing xanthorrhizol, curcumin, immune polysaccharides and insoluble dietary fiber obtained by the said preparing method, and general food, health functional food, cosmetic, pharmaceutical compositions and the like containing the extracts as an active ingredient. Since the Curcuma xanthorrhiza Roxb. extraction method according to the present invention can obtain various kinds of natural extracts from one raw material by applying a variety of extraction methods, the method can be very useful for the production of extracts using natural products.

### TECHNICAL FIELD

The present invention relates to a method for preparing a Curcuma xanthorrhiza Roxb. extract, and to a method for obtaining various functional extracts from Curcuma xanthorrhiza Roxb.

### BACKGROUND ART

Curcuma xanthorrhiza Roxb. is a Zingiberaceae plant which is a traditional medicinal herb, commonly known as temu lawak or Javanese turmeric in Indonesia, and includes terpenoid-based compounds, such as artumenone, α-curcumene, β-curcumene, curzerenone, germacrone, β-sesquiphellandrene, α-turmerone, β-turmerone, xanthorrhizol, etc., 7% to 30% essential oil, 30% to 40% carbohydrates, and 0.02% to 2.0% aromatic pigments such as curcuminoid, etc. (Am. J. Chin. Med. 23: 243-254, 1995).

For example, as the conventional extraction methods, a simple hot-water extraction method as disclosed in Korean Patent Publication No. 2002-0048848 to elute the extraction target in hot water and a method of cooling after extraction while maintaining a specific pressure as disclosed in Korean Patent No. 10-0847806 are known.

In the case of extraction at a low temperature, there is a problem in that it is not possible to efficiently extract components that can be extracted by leaving thereof at high temperature or for a long time, and this may lead to an economical problem of lowering the efficiency of the process.

Since the conventional extraction processes have a limited yield and function by applying a separate single process, there is a limit to the efficient utilization of raw materials.

### DISCLOSURE

### Technical Problem

The present invention is objected to provide a method for preparing a Curcuma xanthorrhiza Roxb. extract.

### Technical Solution

In order to solve the above problem, the present invention provides a method for preparing a Curcuma xanthorrhiza Roxb. extract comprising the following steps of:

1) subjecting Curcuma xanthorrhiza Roxb. to supercritical extraction to prepare the 1^{st} extract;

2) subjecting a residue of the 1^{st} extract to ethanol extraction or supercritical extraction to prepare the 2^{nd} extract;

3) subjecting a residue of the 2^{nd} extract to enzyme treatment to prepare the 3^{rd} extract; and

4) obtaining a residue containing insoluble dietary fiber.

According to one embodiment, the supercritical extraction of the step 1) and step 2) may be conducted using carbon dioxide or pentane as a supercritical fluid at 150 bar to 400 bar and 30° C. to 100° C. for 5 minutes to 240 minutes, to extract xanthorrhizol as an active ingredient.

Further, the supercritical extraction of the step 2) may be conducted using ethanol as a co-solvent, to extract curcumin as an active ingredient.

According to one embodiment, the ethanol extraction may be conducted using ethanol at 40° C. to 120° C. for 0.5 hour to 10 hours, to extract curcumin as an active ingredient.

According to one embodiment, the enzyme treatment may be conducted using at least one enzyme selected from the group consisting of cellulase and pectinase at 30° C. to 90° C. for 0.5 hour to 5 hours, and hydrolyzing the remaining starch using amylase, followed by ultrafiltration thereof.

According to one embodiment, the cellulase and the pectinase may be an enzyme complex comprising at least one selected from the group consisting of cellulase, hemicellulase and pectinase, and the amylase may include α-amylase.

According to one embodiment, the ultrafiltration may use a membrane having a molecular weight cut-off of 500 to 10,000.

According to one embodiment, the Curcuma xanthorrhiza Roxb. extract may contain xanthorrhizol, curcumin and immune polysaccharides.

According to another embodiment, a Curcuma xanthorrhiza Roxb. extract prepared according to the method described above can be provided.

Other specific embodiments of the present invention are included in the following detailed description.

### Advantageous Effects

According to the method for preparing a Curcuma xanthorrhiza Roxb. extract of the present invention, by applying the extraction methods stepwise, a wide variety of natural extracts can be obtained from Curcuma xanthorrhiza Roxb.

### DESCRIPTION OF DRAWINGS

FIG. 1 is a drawing showing a process example of the method for preparing a Curcuma xanthorrhiza Roxb. extract.

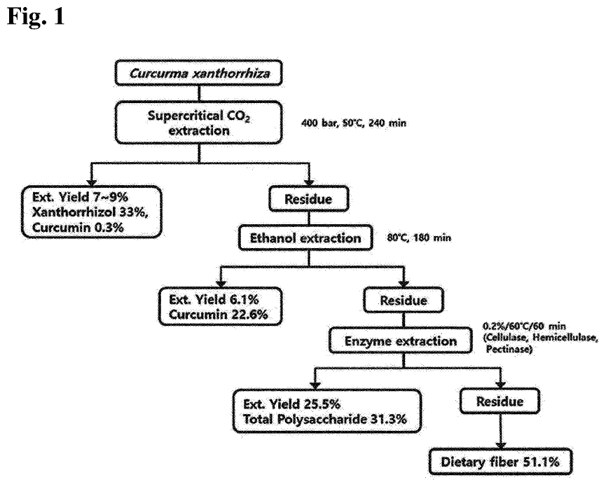

### BEST MODE CARRYING OUT THE INVENTION

The present invention is capable of various modifications and various examples, and specific examples are illustrated in the drawings and described in detail. It should be understood, however, that the invention is not intended to be limited to the particular embodiments, but includes all modifications, equivalents, and alternatives falling within the spirit and scope of the invention. In the following description of the present invention, a detailed description of related arts will be omitted if it is determined that the gist of the present invention may be blurred.

Hereinafter, the method for preparing a Curcuma xanthorrhiza Roxb. extract will be described in more detail.

### MODE FOR INVENTION

The method for preparing a Curcuma xanthorrhiza Roxb. extract according to the present invention may comprise the following steps of:

1) subjecting Curcuma xanthorrhiza Roxb. to supercritical extraction to prepare the 1^{st} extract;

2) subjecting a residue of the 1^{st} extract to ethanol extraction or supercritical extraction to prepare the 2^{nd} extract;

3) subjecting a residue of the 2^{nd} extract to enzyme treatment to prepare the 3^{rd} extract; and

4) obtaining a residue containing insoluble dietary fiber.

The supercritical extraction method is a method of extracting using a supercritical fluid. The supercritical fluid means a fluid having a state exceeding critical temperature and boundary pressure. For example, the extract may be extracted by using at least one solvent selected from the group consisting of water, organic solvents having carbon number of 1 to 6 and subcritical or supercritical fluids. Specifically, the solvent may include at least one selected from water, ethanol, isopropanol, methanol, acetone, ethyl acetate, butanol and hexane. As the supercritical fluid, carbon dioxide, pentane and the like can be used. For example, the method is advantageous in that is can be safely applied to foods or medicines since it proceeds near room temperature using carbon dioxide. Further, a pressure of 150 bar to 400 bar can be applied, and if the pressure is excessively low or high, extraction of the active ingredient may be insufficient. Further, xanthorrhizol can be extracted as an active ingredient from Curcuma xanthorrhiza Roxb. at a the temperature of 30° C. to 100° C., for example, 40° C. to 60° C. and the time of 5 minutes to 240 minutes, for example, 60 minutes to 240 minutes. Further, in the supercritical extraction method of the step 2), curcumin can be extracted as an active ingredient by flowing ethanol as a co-solvent at 1 mL/min to 12 mL/min for 5 minutes to 120 minutes, for example, at 3 mL/min to 10 mL/min for 30 minutes to 120 minutes.

The ethanol extraction method is a method of extracting an active material from the extraction target by adding ethanol at a high temperature.

According to one embodiment, the active ingredient curcumin can be extracted from Curcuma xanthorrhiza Roxb. at the condition of a temperature of 40° C. to 120° C., for example, 50° C. to 100° C. for 0.5 hour to 10 hours, for example, 1 hour to 5 hours.

According to one embodiment, for the enzyme treatment, the enzyme may be selected from cellulase, pectinase and a combination thereof. Further, the enzyme treatment process may be conducted by treatment with the enzyme at 30° C. to 90° C., for example, 50° C. to 70° C. for 0.5 hour to 5 hours, for example, 0.5 hour to 3 hours and treatment with amylase to hydrolyze the remaining starch using amylase, followed by ultrafiltration thereof, to extract the active ingredient. Specifically, the cellulase and the pectinase may be in the form of an enzyme complex comprising at least one selected from the group consisting of cellulase, hemicellulase and pectinase, and the amylase may include α-amylase. Further, the ultrafiltration may use a membrane having a molecular weight cut-off of 500 to 10,000.

According to one embodiment, the present invention may further comprise a step of conducting a solvent extraction method, ultrasonic extraction method and the like, and can further comprise any general extraction method without any particular limitation. The solvent extraction method is to extract active ingredients by dissolving the extraction target using at least one organic solvent. For example, water, alcohol, ether, petroleum ether, benzene, ethyl acetate, chloroform and the like can be used as the extraction solvent. The ultrasonic extraction method allows deep penetration of the extraction solvent to extract heat-sensitive active ingredients at low temperature condition.

According to the present invention, a Curcuma xanthorrhiza Roxb. extracts containing xanthorrhizol, curcumin, immune polysaccharides and the like can be provided. The immune polysaccharides may mean polysaccharides that enhance immune activity, and may specifically mean polysaccharides that can control the function of macrophage and the like. For example, the immune polysaccharides may include polysaccharides of average molecular weight of 33,000 Da with arabinose, galactose, mannose, rhamnose and xylose as constituent monosaccharides.

According to one embodiment, the Curcuma xanthorrhiza Roxb. extract can be provided by conducting the following steps stepwise, and each step may be suitable repeated by those skilled in the art:

1) subjecting Curcuma xanthorrhiza Roxb. to supercritical extraction to prepare the 1^{st} extract;

2) subjecting a residue of the 1^{st} extract to ethanol extraction or supercritical extraction to prepare the 2^{nd} extract;

3) subjecting a residue of the 2^{nd} extract to enzyme treatment to prepare the 3^{rd} extract; and

4) obtaining a residue containing insoluble dietary fiber.

According to one embodiment, the supercritical extraction, ethanol extraction and enzyme treatment steps can be sequentially carried out, whereby each functional material containing different main ingredients can be obtained from one raw material. For example, the supercritical extract can be used as a material mainly composed of xanthorrhizol for oral health, anti-diabetic, anti-cancer, anti-inflammatory, cranial nerve disorder, body fat reduction and anti-wrinkle, the ethanol and supercritical extract can be used as a material mainly composed of curcumin for hangover, bone disease and brain disease, and the product from the enzyme treatment can be used as a material mainly composed of immune polysaccharides for immunity and hangover. Further, its yield is increased compared to a single process.

The sequential stepwise process as described above can be applied not only to Curcuma xanthorrhiza Roxb. but also to various natural products, and various active ingredients can be extracted from one raw material.

Hereinafter, exemplary embodiments of the present invention will be described in detail so that those skilled in the art can easily carry out the present invention. The present invention may, however, be embodied in many different forms and should not be construed as limited to the exemplary embodiments set forth herein.

### Example: Preparation of Curcuma xanthorrhiza Roxb. Extract

### Example 1: Supercritical Extraction

200 g of Curcuma xanthorrhiza Roxb. dried by hot air and milled was extracted with a supercritical carbon dioxide extracting apparatus under the conditions of 50° C. and a pressure of 400 bar with 100% CO₂ for 240 minutes to obtain 16 g of an extract.

### Example 2-1: Ethanol Extraction

10 times (1800 mL) of ethanol was added to 180 g of the residue obtained from Example 1 and heated and extracted at 80° C. for 3 hours using a heating mantle equipped with a reflux condenser. The resulting extract was filtered, concentrated and dried to obtain 11 g of an extract.

### Example 2-2: Supercritical Extraction

Ethanol as a co-solvent was added to 180 g of the residue obtained from Example 1 at a flow rate of 6 mL/min for 1 hour, and then supercritical extraction was conducted with 100% CO₂ under the condition of 50° C. and a pressure of 400 bar for 120 minutes. The resulting extract was filtered, concentrated and dried to obtain 3.1 g of an extract.

### Example 3: Enzyme Treatment

After adding 10 times (1600 mL) of purified water to 160 g of the residue obtained from Example 2-1, an three-enzyme complex of cellulase, hemicellulase and pectinase was added thereto at a concentration of 0.2%, and then the mixture was stirred at pH 6˜7 and a temperature of 60° C. for 1 hour according to the enzyme reaction conditions. The sample was heated at 90° C. for 10 minutes to inactivate the enzymes and then centrifuged at 6,500 rpm for 15 minutes to obtain a supernatant containing polysaccharides. In order to hydrolyze the starch contained in the product of the enzyme treatment obtained above, α-amylase was treated and then the filtrate was ultrafiltered through a membrane having a molecular weight cut off (MWCO) of 1,000 Da (thin channel ultrafiltration system, Amicon TFC-10; Amicon Co., USA). After the ultrafiltration, a solution having a molecular weight of 1,000 Da or more was collected and freeze-dried to obtain 40.8 g of soluble polysaccharides of Curcuma xanthorrhiza Roxb.

### Example 4: Isolation of Insoluble Dietary Fiber

94 g of the residue obtained from Example 3 was precipitated by adding ethanol and then centrifuged to isolate and obtain 48 g of a residue containing insoluble dietary fiber.

### Test Example: Isolation of Active Ingredient

The samples according to each Example were separated by silica gel column chromatography using a mixed solvent of hexane and ethyl acetate, and then subjected to acetylation and deacetylation to separate and purify them into pure single substances. The molecular weight of each substance containing active ingredients was measured by EI-MS, and the structure was analyzed by confirming the functional groups by 1H-NMR spectrum, 13C-NMR spectrum (400 MHz, CDCl3) and IR.

Further, the extraction yield was determined by freeze-drying each extract and the weight thereof was measured. In the case of xanthorrhizol, the sample was diluted with methanol to be within the calibration concentration range and filtered through a 0.45 μm membrane filter (pore size 0.45 μm, Advantec MFS, Japan), and then the content of the active ingredient was analyzed by HPLC (YL 9100 HPLC system). The analysis column was Waters sunfire C₁₈ column (5 μm, 4.6×450 mm, Waters Co) and the mobile phase was 80% methanol (JTbaker Co, Phillipsburg, USA). The flow rate was 1.0 mL/min, injection volume was 20 μL and the detector was used with UV at 280 nm. In the case of curcumin, the sample was diluted with methanol to be within the calibration concentration range and filtered through a 0.45 μm membrane filter (pore size 0.45 μm, Advantec MFS, Japan), and then the content was analyzed by HPLC (YL 9100 HPLC system). The analysis column was Waters sunfire C₁₈ column (5 μm, 4.6×450 mm, Waters Co) and the mobile phase was a mixture of acetonitrile (JTbaker Co, Phillipsburg, USA) and 2% acetic acid at 40:60 ratio. The flow rate was 1.0 mL/min, the injection volume was 20 μL and the detector was used with UV at 425 nm. The immune polysaccharides were cut off by ultrafiltration at MW 1,000 or less, and then the solution of MW 1,000 Da or more were collected and freeze-dried and then analyzed by weighing. The results of analyzing each component are shown in Table 1 below.

TABLE 1

| | Exam- | Exam- | Exam- | Exam- | Exam- |
|---|---|---|---|---|---|
| Section | ple 1 | ple 2-1 | ple 2-2 | ple 3 | ple 4 |
| Extraction Yield (%) | 7~9 | 5.1 | 1.71 | 25.5 | 51.1 |
| Xanthorrhizol (%) | 33 | — | — | — | — |
| Curcumin (%) | 0.3 | 22.6 | 9.65 | — | — |
| Immune | — | — | — | 31.3 | — |
| polysaccharides (%) | | | | | |
| Dietary fiber (%) | — | — | — | — | 100 |

As can be seen from the above results, it can be found that various active ingredients can be effectively extracted from Curcuma xanthorrhiza Roxb. according to the method for preparing a Curcuma xanthorrhiza Roxb. Extract according to the present invention.

The above description is only illustrative of the technical idea of the present invention, and various changes and modifications may be made by those skilled in the art without departing from the essential characteristics of the present invention. It should be noted that the embodiments disclosed in the present invention are not intended to limit the scope of the present invention and are not intended to limit the scope of the present invention. It is intended that the present invention cover the modifications and variations of this invention provided they come within the scope of the appended claims and their equivalents.

1. A method for preparing a Curcuma xanthorrhiza Roxb. extract comprising the following steps of:
1) subjecting Curcuma xanthorrhiza Roxb. to supercritical extraction to prepare the 1^{st} extract;
2) subjecting a residue of the 1^{st} extract to ethanol extraction or supercritical extraction to prepare the 2^{nd} extract;
3) subjecting a residue of the 2^{nd} extract to enzyme treatment to prepare the 3^{rd} extract; and
4) obtaining a residue containing insoluble dietary fiber.

2. The method for preparing a Curcuma xanthorrhiza Roxb. extract according to claim 1, wherein the supercritical extraction of the step 1) and step 2) is conducted using carbon dioxide or pentane as a supercritical fluid at a pressure of 150 bar to 400 bar and a temperature of 30° C. to 100° C. for 5 minutes to 240 minutes, to extract xanthorrhizol as an active ingredient.

3. The method for preparing a Curcuma xanthorrhiza Roxb. extract according to claim 2, wherein the supercritical extraction of the step 2) is conducted using ethanol as a co-solvent, to extract curcumin as an active ingredient.

4. The method for preparing a Curcuma xanthorrhiza Roxb. extract according to claim 1, wherein the ethanol extraction is conducted using ethanol at a temperature of 40° C. to 120° C. for 0.5 hour to 10 hours, to extract curcumin as an active ingredient.

5. The method for preparing a Curcuma xanthorrhiza Roxb. extract according to claim 1, wherein the enzyme treatment is conducted using at least one enzyme selected from the group consisting of cellulase and pectinase at a temperature of 30° C. to 90° C. for 0.5 hour to 5 hours, and hydrolyzing starch using amylase, followed by ultrafiltration thereof, to extract polysaccharides as an active ingredient.

6. The method for preparing a Curcuma xanthorrhiza Roxb. extract according to claim 5, wherein the cellulase and the pectinase are in the form of an enzyme complex comprising at least one selected from the group consisting of cellulase, hemicellulase and pectinase, and the amylase includes α-amylase.

7. The method for preparing a Curcuma xanthorrhiza Roxb. extract according to claim 1, wherein the Curcuma xanthorrhiza Roxb. extract contains xanthorrhizol, curcumin and immune polysaccharides.

8. A Curcuma xanthorrhiza Roxb. extract prepared according to the method of claim 1.

9. A Curcuma xanthorrhiza Roxb. extract prepared according to the method of claim 2.

10. A Curcuma xanthorrhiza Roxb. extract prepared according to the method of claim 3.

11. A Curcuma xanthorrhiza Roxb. extract prepared according to the method of claim 4.

12. A Curcuma xanthorrhiza Roxb. extract prepared according to the method of claim 5.

13. A Curcuma xanthorrhiza Roxb. extract prepared according to the method of claim 6.

14. A Curcuma xanthorrhiza Roxb. extract prepared according to the method of claim 7.
